(19) United States
(12) Patent Application Publication
(10) Pub. No.: US 2008/0287852 A1
(43) Pub. Date: Nov. 20, 2008

(54) WATER RESISTANT ELASTICIZED RETENTION BANDAGE AND UNDERCAST LINER

(76) Inventor: John C. Evans, Lancashire (GB)

Correspondence Address: ADAMS INTELLECTUAL PROPERTY LAW, P.A., Suite 2350 Charlotte Plaza, 201 South College Street, CHARLOTTE, NC, 28244, US

(21) Appl. No.: 12/121,165

(22) Filed: May 15, 2008

Related U.S. Application Data
(60) Provisional application No. 60/938,505, filed on May 17, 2007.

(51) Int. Cl. A61F 13/00

(52) U.S. Cl. 602/43

(57) ABSTRACT

A fabric substrate suitable for lining between a cast and a person. The fabric substrate includes a pair of opposing faces formed by pillar and inlay stitches. The fabric is constructed at least in part of hydrophobic, water-resistant yarns configured to provide enhanced water resistance and sufficient stretch in both a length and width direction to facilitate conforming the undercast liner around the anatomical shape without wrinkling during application. An intermediate tying layer spans between the opposing faces, and the tying layer includes elastic yarns stitched in a V stitch pattern such that the elastic yarns extend between one opposing fabric face and the other face.

### CROSS REFERENCE TO RELATED APPLICATION

This application claims priority to U.S. Provisional Patent Application No. 60/938,505 which was filed on May 17, 2007.

### TECHNICAL FIELD AND BACKGROUND OF THE INVENTION

The invention relates to water resistant medical bandages and more particularly to a water resistant elasticized bandage and undercast liner.

Current bandages are constructed from woven or knitted fabrics using natural or synthetic yarns. These yarns are difficult to dry once wet and generally have poor air permeability and limited water resistance. The bandage may be used to secure a splint, cast, padding, or swab against the skin for extended periods of time. Because of the poor air permeability of current bandages, this can lead to poor skin conditions such as maceration of the skin, bacteria growth, and odors.

Accordingly, there is a need for a bandage or cast liner that has sufficient porosity and strength so as to not cause skin maceration, bacteria growth, and odorous conditions.

### SUMMARY OF THE INVENTION

Therefore, it is an object of the invention to provide a water-resistant elastic retention bandage.

It is another object of the invention to provide a water-resistant cast liner.

It is another object of the invention to provide a cast liner having improved bacterial prevention characteristics.

These and other objects of the present invention are achieved in the preferred embodiments disclosed below by providing a fabric substrate suitable for lining between a cast and a person. The fabric substrate includes a pair of opposing faces formed by pillar and inlay stitches. The fabric is constructed at least in part of hydrophobic, water-resistant yarns configured to provide enhanced water resistance and sufficient stretch in both a length and width direction to facilitate conforming the undercast liner around the anatomical shape without wrinkling during application. An intermediate tying layer spans between the opposing faces. The tying layer includes elastic yarns stitched in a V stitch pattern such that the elastic yarns extend between the opposing faces.

According to another embodiment of the invention, the substrate includes an adhesive coating on the opposing faces.

According to another embodiment of the invention, the adhesive is a low tack pressure sensitive adhesive selected from the group consisting of acrylic adhesives, silicone adhesives, and combinations thereof.

According to another embodiment of the invention, the tying layer includes non-elastic yarns stitched in a V stitch pattern such that the non-elastic yarns extend from one opposing fabric face to the other face, and the non-elastic yarn is comprised of a material selected from the group consisting of polypropylene, nylon, polyethylene, and combinations thereof.

According to another embodiment of the invention, the diameter of the non-elastic yarn is greater than 0.03 mm.

According to another embodiment of the invention, the V stitch pattern is a 3 needle V stitch pattern.

According to another embodiment of the invention, the V stitch pattern is a 5 needle V stitch pattern.

According to another embodiment of the invention, the substrate is formed such that it has at least 50 courses per meter and weighs between about 50 and about 400 grams per square meter.

According to another embodiment of the invention, the substrate weighs between about 100 and about 250 grams per square meter and has a nominal thickness when not compressed or under tension of between about 1.5 and about 3.5 mm.

According to another embodiment of the invention, the substrate is treated with at least one of a fluorochemical, silicone, other water repellant finish, and combinations thereof for improving drainage and providing faster drying.

According to another embodiment of the invention, an undercast liner for being applied to an anatomical shape of a patient and overlaid with a cast material is provided. The undercast liner includes a tubularfabric having a pair of opposing circumferential faces formed by pillar and inlay stitches. The fabric is constructed at least in part of hydrophobic, water-resistant yarn for providing enhanced water resistance, light weight, breathability and resistance to collapse and degradation due to moisture and bacteria during extended use of the liner. The fabric has sufficient stretch in both a length and width direction to facilitate conforming the undercast liner around the anatomical shape during application. An intermediate tying area provided between the opposing circumferential faces ties the pair of opposing faces together. The tying area includes elastic yarns stitched in a V stitch pattern such that the elastic yarns extend from one opposing face to the other face.

According to an embodiment of the invention, the spacer area includes non-elastic yarns.

According to an embodiment of the invention, the non-elastic yarns are between about 0.003 and about 1.55 mm in diameter.

According to an embodiment of the invention, the fabric includes a water repellant finish to improve drainage and provide faster drying.

According to another embodiment of the invention, a retention bandage in roll form for being applied to an anatomical shape of a patient is provided. The bandage includes an elongate fabric having a pair of opposing faces formed by pillar and inlay stitches. The fabric is constructed at least in part of hydrophobic, water-resistant monofilament yarns of about 33 to about 78 Decitex configured to provide enhanced water resistance and breathability during extended use of the liner. The fabric has sufficient stretch in both a length and width direction to facilitate conforming the undercast liner around the anatomical shape during application. An intermediate tying layer is provided between the opposing faces. The tying layer includes elastic yarns stitched in a V stitch pattern such that the yarns extend from one opposing fabric face to the other face.

According to another embodiment of the invention the bandage is between about 0.5 to about 3.0 mm thick.

According to another embodiment of the invention, the bandage is selected from the group consisting of polypropylene, polyester, nylon, polymethane, and combinations thereof.

According to another embodiment of the invention, the monofilament yarns have a diameter of between about 0.05 and about 0.25 mm.

According to another embodiment of the invention, the elastic yarn has a Decitex of between about 33 and about 150.

According to another embodiment of the invention, the bandage weighs between about 100 to 250 grams per square meter and has a nominal thickness of between about 1.5 and about 3.5 mm.

According to another embodiment of the invention, the monofilament yarns may be between about 0.03 mm and about 1.5 mm in diameter.

According to another embodiment of the invention, the substrate may have a thickness of between about 0.5 and about 10 mm.

According to another embodiment of the invention, the substrate weight may be between about 40 and about 160 grams per square meter.

According to another embodiment of the invention, the substrate may have between about 4 and about 20 courses per inch.

According to another embodiment of the invention, the substrate is between about 6 and about 28 wales per inch.

According to another embodiment of the invention, the substrate may contain a multifilament yarn.

According to another embodiment of the invention, the substrate is used in a bandaging product or in a compression bandage, and incorporates elastic yarns.

### BRIEF DESCRIPTION OF THE DRAWINGS

Some of the objects of the invention have been set forth above. Other objects and advantages of the invention will become apparent when the description thereof is taken in conjunction with the following drawings, in which:

FIG. 1 is a perspective view of a roll of an undercast liner according to one embodiment of the invention;

FIG. 2 is a view illustrating application of the undercast liner to the wrist and forearm;

FIGS. 3 and 4 illustrate preparation of a cast tape for application over the undercast liner;

FIGS. 5 and 6 illustrate application of the cast tape to the undercast liner;

FIG. 7 illustrates the completed cast;

FIG. 8 illustrates a liner in the form of a tubular sleeve;

FIG. 9 shows the sleeve in place on an arm prior to application of a cast tape bandage;

FIG. 10 is a perspective view of a water-resistant elasticized retention bandage according to a preferred embodiment of the invention;

FIG. 11 is another perspective view of the retention bandage of FIG. 10;

FIG. 12 is yet another perspective view of the retention bandage of FIG. 10;

FIGS. 13 and 14 illustrate the retention bandage of FIG. 10 being used to secure a splint in position against the anatomy of a patient; and

FIG. 14 illustrates the retention bandage in a rolled form.

### DESCRIPTION OF THE PREFERRED EMBODIMENTS AND BEST MODE

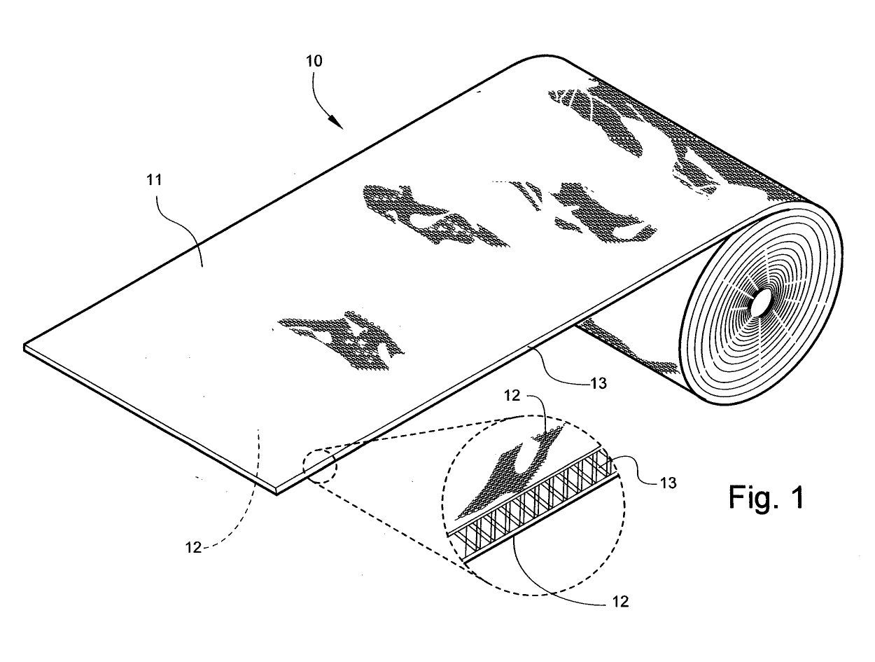

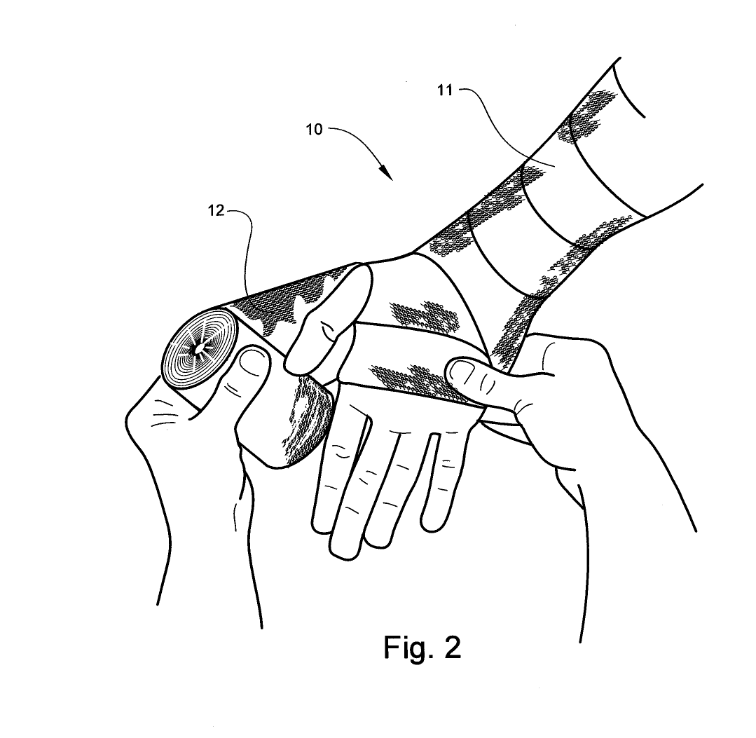

Referring now specifically to the drawings, a undercast liner according to the present invention is illustrated in FIG. 1 and shown generally at reference numeral 10. The undercast liner 10 is easily dispensed from the roll, as shown in FIG. 2. The liner 10 includes two opposing faces 11, 12 and an intermediate spacer area 13 that both separates and interconnects the faces 11, 12, as described in further detail below. The undercast liner 10 can be formed in a tubular form and formed in a roll.

The liner 10 can be constructed using any suitable organic or inorganic monofilament yarn, preferably a hydrophobic/water-resistant monofilament yarn such as polypropylene, polyester, polyethylene, and nylon. The monofilament yarn used for constructing the liner 10 preferably has a diameter of at least 0.03 mm. The liner 10 is constructed in a spacer fabric construction to provide sufficient cushioning and breathability. The use of a monofilament hydrophobic yarn on both faces 11, 12 and in the spacer area 13 provides enhanced water resistance, light weight, breathability, and resistance to collapse and degradation due to exposure to moisture and bacteria during extended use.

The liner 10 is formed using any suitable fabric forming technology such as weaving, various knitting techniques such as, for example, weft knitting and warp knitting, non-woven, stitching, or a combination of these techniques. Preferably, the structure should provide some stretch in both the length and width directions, and facilitate conforming the undercast liner 10 around an anatomical shape during application.

The liner 10 can be treated with one or more finishes to provide additional water resistance, anti-bacterial, anti-odor characteristics, or aromatherapy to improve the functionality or enhance the cast-wearing experience for the patient. Alternatively, the liner 10 can be fabricated from modified or treated monofilament yarns incorporating suitable fillers or finishes to improve the performance of the liner 10.

The liner 10 may also be provided with an adhesive coating on one or both faces 11, 12 to aid in application to the patient. The adhesive is preferably any suitable low tack, pressure sensitive adhesive, such as an acrylic or silicone adhesive. The adhesive aids in application by adhering to itself and thus maintaining the exact placement of the layers relative to each other as the liner 10 is applied by the cast technician.

In one embodiment, the liner 10 is constructed as a spacer fabric using polypropylene monofilament and a low tack, pressure sensitive adhesive on one surface. The monofilament yarn has a diameter of at least 0.03 mm, and preferably between about 0.05 and about 0.25 mm. Preferably, the liner 10 requires no additional finish or water repellency treatment.

More specifically, the liner 10 is constructed of a polypropylene monofilament yarn on a double needle bed knitting machine, and can be knitted on either a warp knitting Raschel machine or a Crochet knitting machine. The liner 10 is preferably constructed using a pillar and inlay stitch on the surfaces 11, 12 and a 3 or 5 needle V in the spacer area 13. The yarn has a diameter of between about 0.03 and about 0.25 mm. The fabric for the liner 10 is formed with at least about 50 courses per meter and preferably between about 200 and about 850 courses per meter. The liner 50 weighs between about 50 and about 400 grams per square meter, and more preferably between about 100 and about 250 grams per square meter. The liner 10 has a nominal thickness when not compressed or under tension of between about 1.5 and about 3.5 mm.

Alternatively, an undercast liner may be constructed as a spacer fabric with at least one of the yarns being a multifilament or spun yarn in order to provide even more patient comfort. The liner can be treated with a suitable fluorochemical, silicone, or other water repellant finish to improve drainage and provide faster drying.

Referring now to FIG. 2, the undercast liner 10 is applied to the injured limb in a conventional manner. As noted above, the stretch provided by the undercast liner 10 permits a fast, accurate, closely-conforming application without wrinkles or creases.

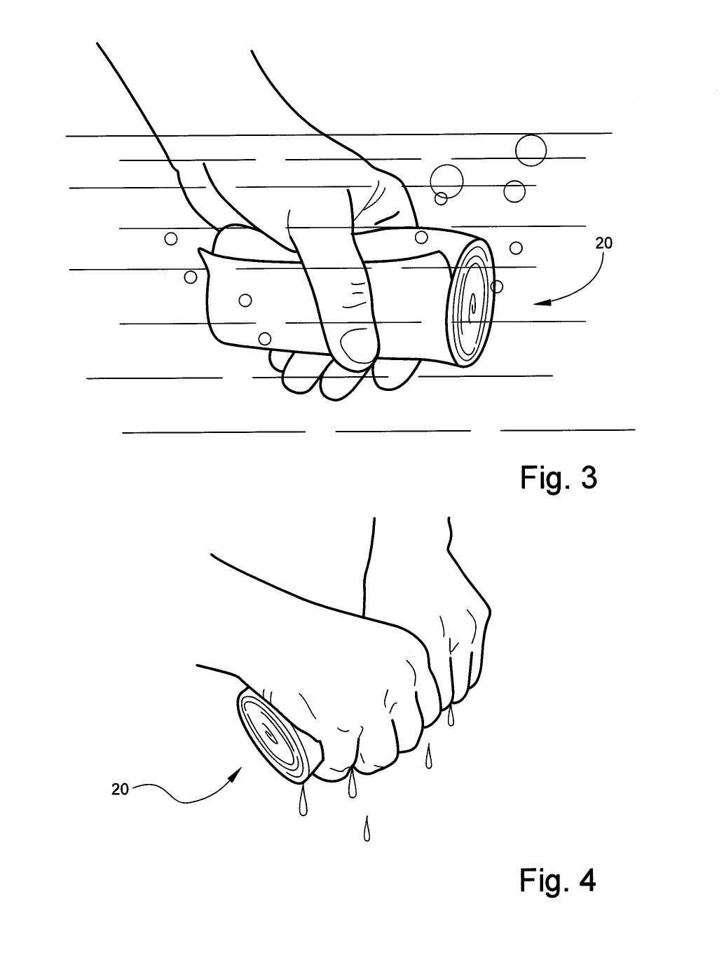

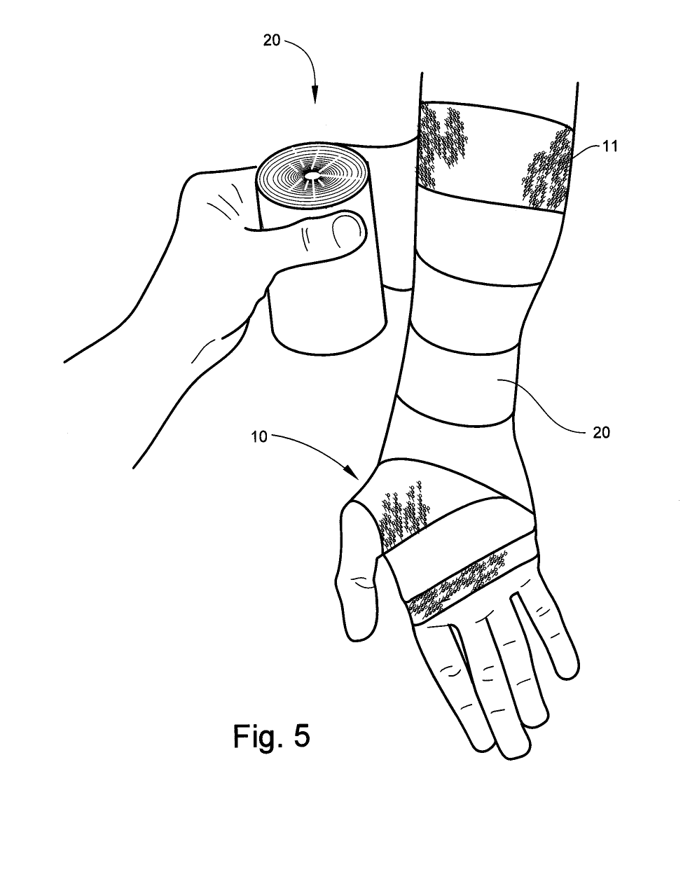

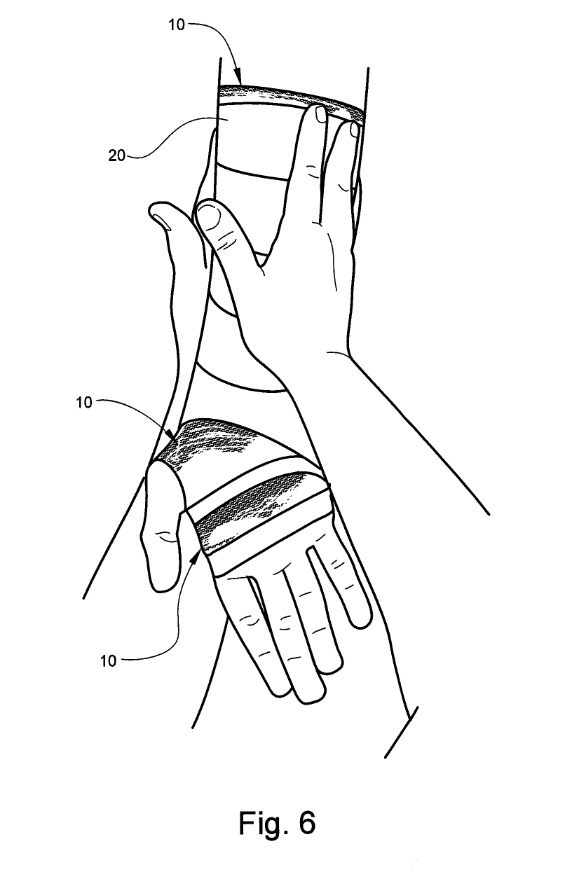

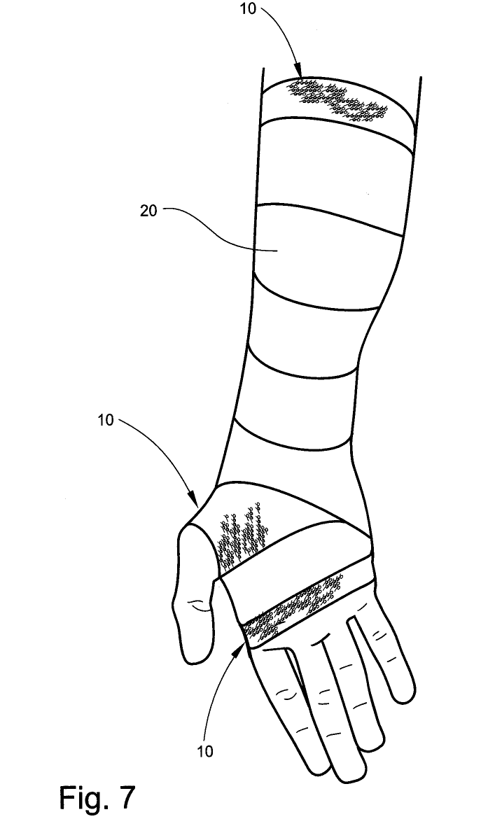

As is shown in FIGS. 3-7, after application of the undercast liner 10, a conventional cast tape 20 is wetted, FIG. 3, excess water removed by wringing, FIG. 4, and applied to the injured limb, FIGS. 5-7, taking care in the conventional manner to avoid overlapping the undercast liner 10 on opposite ends, leaving a short width of exposed undercast liner 10.

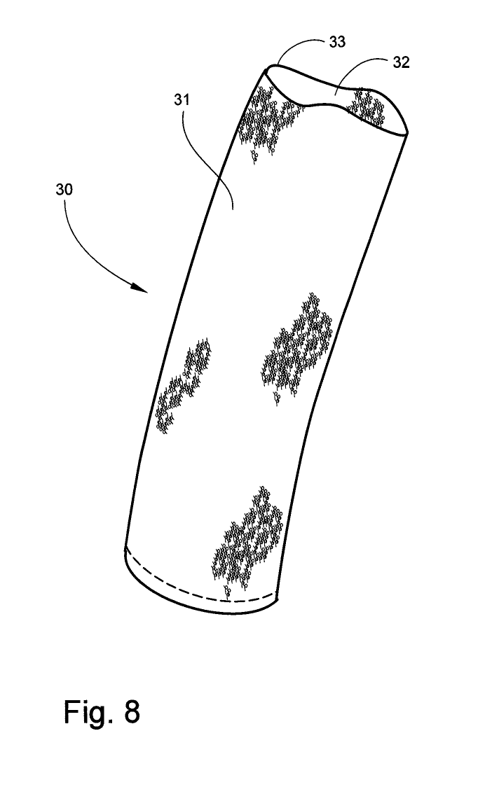

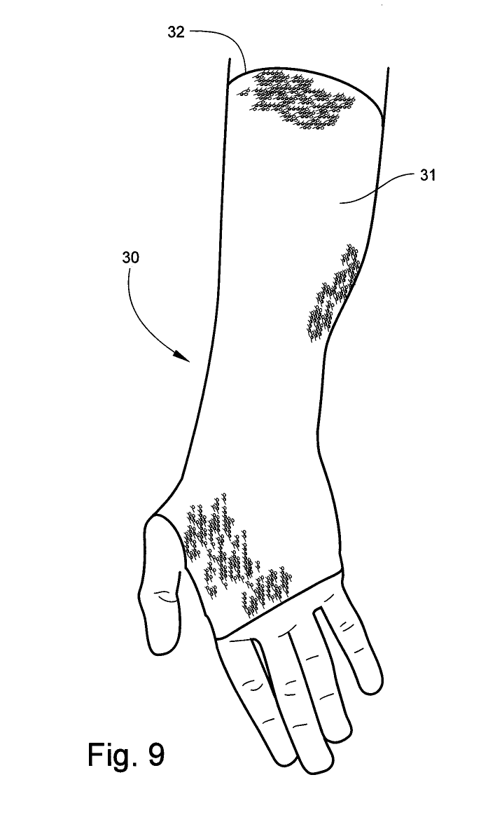

Referring now to FIG. 8, a circular knit liner 30 is shown, preferably with the same constructions described above. The liner 30 includes an outer face 31, an inner face 32, and a spacer area 33. The spacer are 33 both separates and interconnects the two faces 31, 32, as shown in FIG. 1. Instead of wrapping, the liner 30 is pulled onto the limb as shown in FIG. 9, in the same manner as a conventional stockinette. Thereafter, a cast tape 20 is applied in a conventional manner.

A further embodiment includes a knitted spacer fabric constructed from monofilament yarns. The monofilament yarns may be of the type selected from Nylon, Polypropylene, Polyester yarns or a mixture thereof.

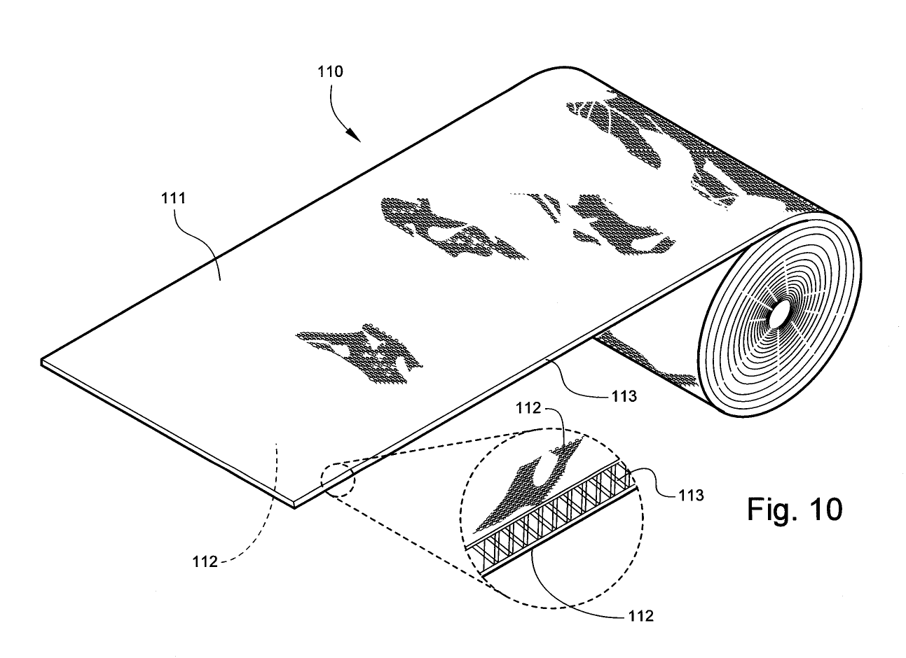

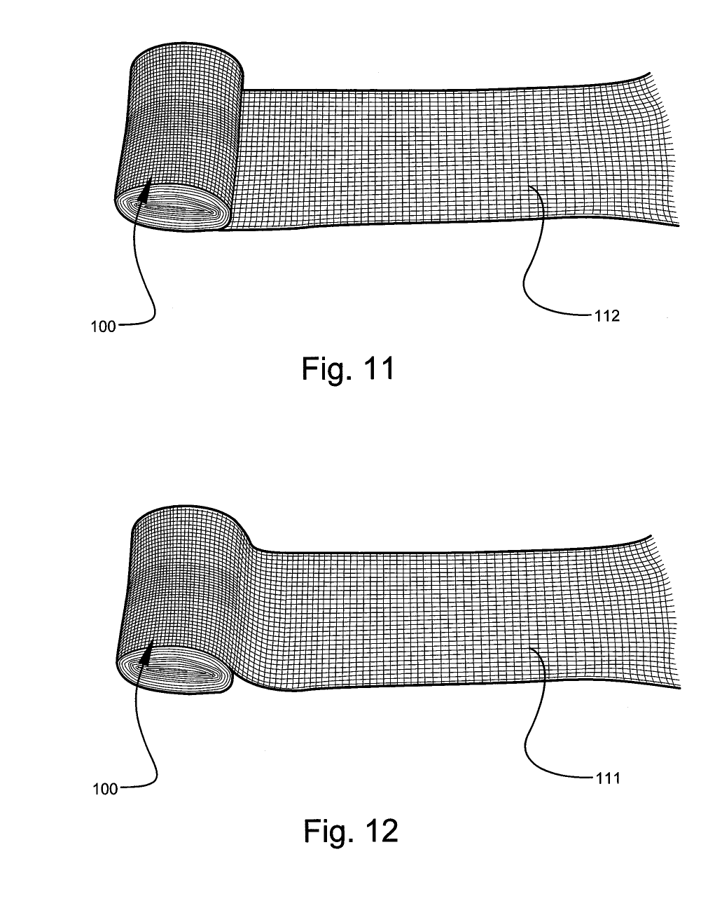

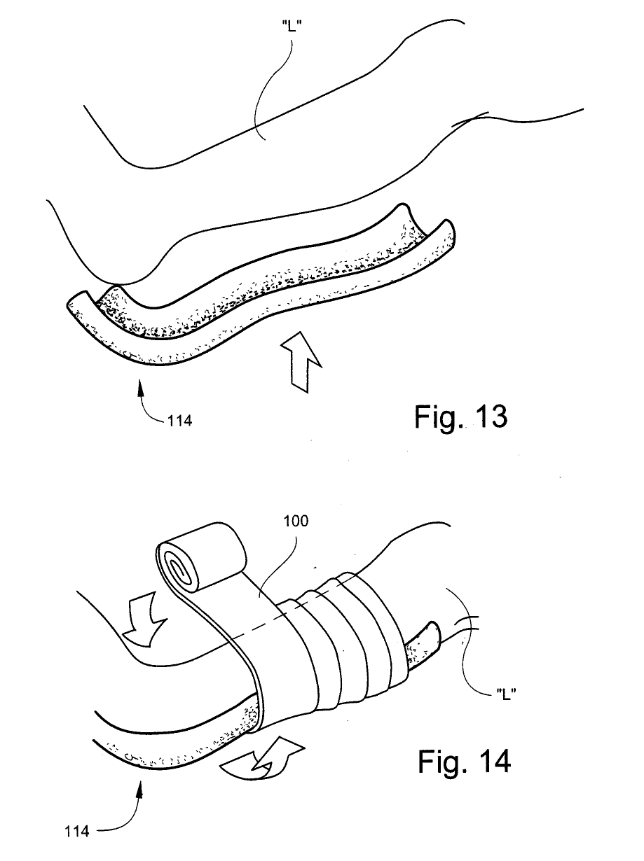

A water-resistant elasticized retention bandage according to a preferred embodiment of the invention is illustrated in FIGS. 10-13, and shown generally at reference numeral 100. As shown in FIG. 10, the retention bandage 10 comprises an elongate knitted fabric having two opposing surfaces 111, 112 and an intermediate spacer section 113. The spacer are 113 both separates and connects the opposed surfaces 111, 112. The elastic retention bandage 100 is constructed at least in part of hydrophobic, water-resistant monofilament yarns to create an efficient breathable, porous, lightweight structure that can dry rapidly. The retention bandage 100 can be stretched sufficiently in both width and length directions to provide excellent application aspects that avoids creasing and wrinkling when applied to an anatomical shape. As shown in FIGS. 13 and 14, the retention bandage 100 can be applied to an anatomical shape of a patient such as a leg “L” to hold in position an orthopedic device such as a splint 114.

Preferably, the retention bandage 100 can be stretched to a greater extent in the length direction than in the width direction in order to give more compressive force to hold the splint 114, or other medical device such as a swab or padding, in position without slippage.

An adhesive such as a hot melt adhesive can be applied to one surface of the bandage substrate to tack the material in position and prevent movement of the bandage 100. The adhesive permits adhesion of the bandage 100 to itself and prevents movement of the bandage 100 when in situ supporting or holding in position the splint or padding or swab. The adhesive mass is preferably based on a weight of between about 2 and about 30 grams per square meter, and is preferably of a type that is skin friendly. Alternatively, the bandage 10 can be used without an adhesive, and the end of the bandage 10 can be secured by a clip, pin, sticky tape, hook and loop fasteners, and combinations thereof.

The knitted spacer fabric 113 is constructed of highly resilient monofilament yarns combined with elastic yarns that give a stretch capability to the structure to aid and support application to the anatomical structure. The use of the monofilament yarns ensures effective water drainage and drying due to very low surface area and surface energy. The yarns used in the elastic retention bandage 100 are preferably polypropylene, polyester, nylon, polyurethane and combinations thereof, and the fabric of the bandage 100 can be formed by weaving, knitting or other suitable method.

The retention bandage 100 is preferably constructed using a pillar, inlay, and ‘V’ needle structure in the center stitch. The bandage 100 is a lightweight structure and preferably weighs between about 50 and about 200 grams per square meter. The invention can provide a more comfortable, conformable, breathable, water-resistant bandage 100 at a lower cost than other products available in the market. The present invention's use of a novel construction of monofilament yarns yields excellent padding and openness that permits safer application and air circulation around the injury site. The substrate has a relatively high moisture vapor transmission rate of about 1500 grams per day per square meter.

The invention can significantly improve air circulation leading to improvements in skin condition and maceration levels. The present invention allows the patient to swim, bath or shower and keep the injury site clean and healthy without damaging the support or retention bandage.

A water-resistant elasticized retention bandage and a water-resistant undercast liner are described above. Various details of the invention may be changed without departing from its scope. Furthermore, the foregoing description of the preferred embodiments of the invention and the best mode for practicing the invention are provided for the purpose of illustration only and not for the purpose of limitation.

I claim:

1. A fabric substrate suitable for lining between a cast and a person comprising:
(A) a pair of opposing faces formed by pillar and inlay stitches, wherein the fabric is constructed at least in part of hydrophobic, water-resistant yarns configured to provide enhanced water resistance and to provide sufficient stretch in both a length and width direction to facilitate conforming the undercast liner around the anatomical shape without wrinkling during application; and
(B) an intermediate tying layer between the opposing faces, wherein the tying layer includes elastic yarns stitched in a V stitch pattern such that the elastic yarns extend between one opposing fabric face and the other face.

2. The substrate according to claim 1, further including an adhesive coating on the opposing faces.

3. The substrate according to claim 2, wherein the adhesive is a low tack pressure sensitive adhesive selected from the group consisting of acrylic adhesives, silicone adhesives, and combinations thereof.

4. The substrate according to claim 1, wherein the tying layer further includes non-elastic yarns stitched in a V stitch pattern such that the non-elastic yarns extend from one opposing fabric face to the other face, and the non-elastic yarn is comprised of a material selected from the group consisting of polypropylene, nylon, polyethylene, and combinations thereof.

5. The substrate according to claim 4, wherein the diameter of the non-elastic yarn is greater than 0.03 mm.

6. The substrate according to claim 1, wherein the V stitch pattern is a 3 needle V stitch pattern.

7. The substrate according to claim 1, wherein the V stitch pattern is a 5 needle V stitch pattern.

8. The substrate according to claim 1, wherein the substrate is formed such that it has at least 50 courses per meter and weighs between about 50 and about 400 grams per square meter.

9. The substrate according to claim 8, wherein the substrate weighs between about 100 and about 250 grams per square meter and has a nominal thickness when not compressed or under tension of between about 1.5 and about 3.5 mm.

10. The substrate according to claim 1, wherein the substrate is treated with at least one of a fluorochemical, silicone, other water repellant finish, and combinations thereof for improving drainage and providing faster drying.

11. An undercast liner for being applied to an anatomical shape of a patient and overlaid with a cast material, comprising:
(A) a tubular fabric having a pair of opposing circumferential faces formed by pillar and inlay stitches, wherein the fabric is constructed at least in part of hydrophobic, water-resistant yarn for providing enhanced water resistance, light weight, breathability and resistance to collapse and degradation due to moisture and bacteria during extended use of the liner, and the fabric has sufficient stretch in both a length and width direction to facilitate conforming the undercast liner around the anatomical shape during application; and
(B) an intermediate tying area between the opposing circumferential faces, wherein the tying area includes elastic yarns stitched in a V stitch pattern such that the elastic yarns extend from one opposing face to the other face.

12. The undercast liner according to claim 11, wherein the tying layer further includes non-elastic yarns stitched in a V stitch pattern such that the non-elastic yarns extend from one opposing fabric face to the other, and the non-elastic yarn is comprised of a material selected from the group consisting of polypropylene, nylon, polyethylene, and combinations thereof.

13. The undercast liner according to claim 12, wherein the non-elastic yarns are between about 0.003 and about 1.55 mm in diameter.

14. The undercast liner according to claim 11, wherein the fabric includes a water repellant finish to improve drainage and provide faster drying.

15. A retention bandage in roll form for being applied to an anatomical shape of a patient is provided, comprising:
(A) an elongate fabric, having a pair of opposing faces formed by pillar and inlay stitches, wherein the fabric is constructed at least in part of hydrophobic, water-resistant monofilament yarns of between about 33 and about 78 Decitex configured to provide enhanced water resistance and breathability during extended use of the liner, and the fabric has sufficient stretch in both a length and width direction to facilitate conforming the undercast liner around the anatomical shape during application; and
(B) an intermediate tying layer between the opposing faces, wherein the tying layer includes elastic yarns stitched in a V stitch pattern such that the yarns extend from one opposing fabric face to the other face.

16. The retention bandage according to claim 15, wherein the bandage is between about 0.5 and about 3.0 mm thick.

17. The retention bandage according to claim 15, wherein the bandage is selected from the group consisting of polypropylene, polyester, nylon, polymethane and combinations thereof.

18. The retention bandage according to claim 15, wherein the monofilament yarns have a diameter of between about 0.05 and about 0.25 mm.

19. The retention bandage according to claim 15, wherein the elastic yarn has a Decitex of between about 33 and about 150.

20. The retention bandage according to claim 15, wherein the bandage weighs between about 100 and about 250 grams per square meter and has a nominal thickness of between about 1.5 and about 3.5 mm.
